(19) United States
(12) Patent Application Publication
(10) Pub. No.: US 2026/0337136 A1
(43) Pub. Date: Oct. 29, 2026

(54) Method of Treating an Ethylenically Unsaturated Halogenated Hydrocarbon

(71) Applicant: Westlake US 2 LLC, Houston, TX (US)

(72) Inventors: Rahul Patel, Baton Rouge, LA (US); Scott A. Sherwood, Irwin, PA (US)

(21) Appl. No.: 19/653,264

(22) Filed: Apr. 21, 2026

Related U.S. Application Data
(60) Provisional application No. 63/794,408, filed on Apr. 25, 2025.

(51) Int. Cl. C07C 17/395; C07C 21/06

(52) U.S. Cl. CPC C07C 17/395; C07C 21/06

(57) ABSTRACT

The present invention relates to a method of treating a halogenated hydrocarbon that involves providing a first composition including a halogenated hydrocarbon that includes at least one halogen group, where at least one halogen group is covalently bonded to an sp² hybridized carbon, and where the first composition has a first hydrogen halide content. The method additionally involves contacting together the first composition and a hydrotalcite material to form a second composition that includes the halogenated hydrocarbon and a second hydrogen halide content that is less than the first hydrogen halide content. The second composition is isolated from the hydrotalcite material.

### CROSS-REFERENCE TO RELATED APPLICATION

This Nonprovisional patent application is entitled to and claims priority to U.S. Provisional Patent Application No. 63/794,408, which was filed on Apr. 25, 2025, the disclosure of which is incorporated herein by reference in its entirety.

### FIELD OF THE INVENTION

The present invention relates to a method of treating a halogenated hydrocarbon that involves providing a first composition including a halogenated hydrocarbon that includes at least one halogen group, where at least one halogen group is covalently bonded to an sp² hybridized carbon, where the first composition has a first hydrogen halide content, contacting together the first composition and a hydrotalcite material to form a second composition that includes the halogenated hydrocarbon and a second hydrogen halide content that is less than the first hydrogen halide content, and isolating the second composition from the hydrotalcite material.

### BACKGROUND OF THE INVENTION

Halogenated ethylenically unsaturated hydrocarbons are often contaminated with hydrogen halide. The hydrogen halide contamination can result from processes by which the ethylenically unsaturated hydrocarbons (and/or their unsaturated precursors) are halogenated, such as free radical halogenation processes. In addition, or alternatively, halogenated ethylenically unsaturated hydrocarbons can undergo decomposition reactions, such as under conditions of elevated temperature and/or contact with metal salts, which results in the formation of hydrogen halides. The presence of hydrogen halides in the halogenated ethylencially unsaturated hydrocarbon can lead to problems with further processing, such as corrosion of storage and processing equipment, and/or degraded properties, such as discoloration and/or reduced physical properties, of downstream products.

Residual hydrogen halides, such as hydrogen chloride, can be removed by treatment with alkali metal hydroxides, such as sodium hydroxide, and/or alkaline earth metal hydroxides. Such treatment methods can be problematic in that the hydroxides, such as sodium hydroxide, themselves have corrosive properties associated therewith. In addition, treatment with hydroxide results in the formation of salts, which typically must be removed by additional process steps, such as aqueous washing.

It would be desirable to develop new methods of removing hydrogen halides from halogenated ethylenically unsaturated hydrocarbons. It would be further desirable that such newly developed methods do not involve the use of toxic or corrosive chemicals.

### SUMMARY OF THE INVENTION

In accordance with the present invention, there is provided a method of treating a halogenated hydrocarbon that comprises (a) providing a first composition that comprises a halogenated hydrocarbon comprising at least one halogen group, wherein at least one halogen group is covalently bonded to an sp² hybridized carbon, wherein the first composition has a first hydrogen halide content (or a first amount of hydrogen halide). The method further comprises (b) contacting together the first composition and a hydrotalcite material, thereby forming a second composition comprising the halogenated hydrocarbon and having a second hydrogen halide content (or a second amount of hydrogen halide). The second hydrogen halide content is less than the first hydrogen halide content. The method also comprises (c) isolating the second composition from the hydrotalcite material.

In further accordance with the present invention there is provided a method of treating a halogenated hydrocarbon that comprises: (a) providing a feed stream that comprises a first composition comprising a halogenated hydrocarbon comprising at least one halogen group, wherein at least one halogen group is covalently bonded to an sp² hybridized carbon, and wherein said first composition has a first hydrogen halide content (or a first amount of hydrogen halide); and (b) providing a first fixed bed vessel comprising a first fixed bed comprising a hydrotalcite material. The method further comprises (c) introducing the feed stream into the first fixed bed vessel, and contacting together within the first fixed bed vessel the first composition and the hydrotalcite material. The method also comprises (d) withdrawing a product stream from the first fixed bed vessel, wherein the product stream comprises a second composition comprising the halogenated hydrocarbon having a second hydrogen halide content (or a second amount of hydrogen halide). The second hydrogen halide content is less than the first hydrogen halide content.

The features that characterize the present invention are pointed out with particularity in the claims, which are annexed to and form a part of this disclosure. These and other features of the invention, its operating advantages and the specific objects obtained by its use will be more fully understood from the following detailed description in which non-limiting embodiments of the invention are illustrated and described.

### BRIEF DESCRIPTION OF THE DRAWINGS

FIG. 1 is a schematic representation of a process assembly that includes a first fixed bed vessel, which can be used in the method of the present invention; and

FIG. 2 is a schematic representation of a process assembly that includes a first fixed bed vessel and a second fixed bed vessel, which can be used in the method of the present invention.

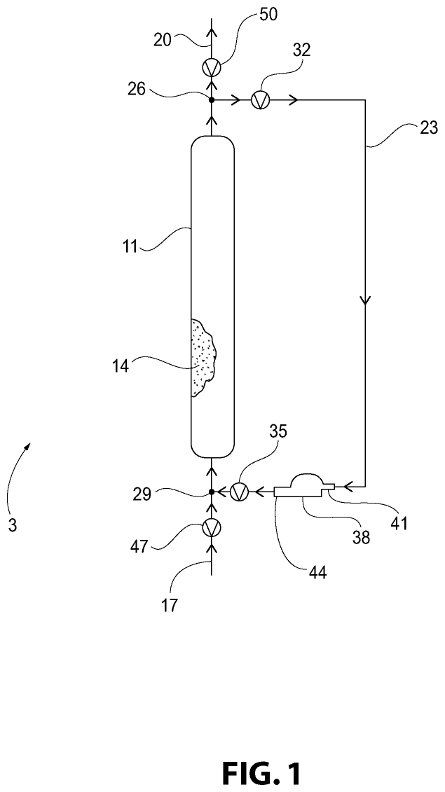

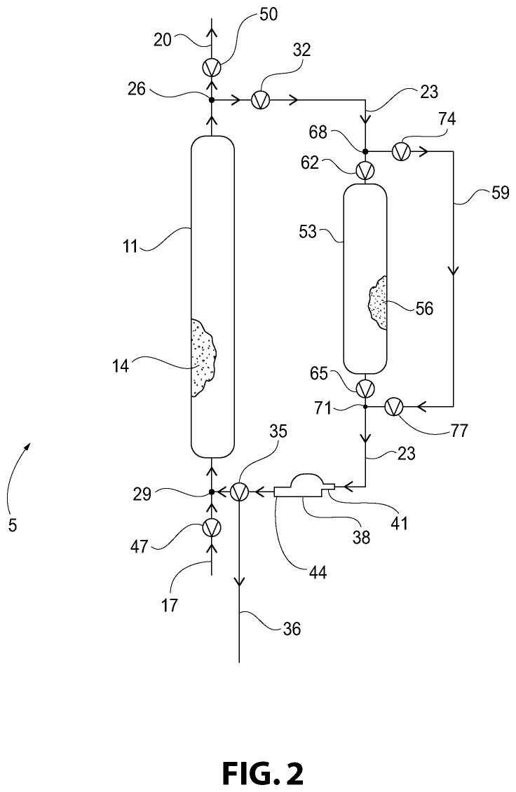

In FIGS. 1 and 2, which are not to scale, like reference characters designate the same components and structural features.

### DETAILED DESCRIPTION OF THE INVENTION

As used herein, spatial or directional terms, such as “left”, “right”, “inner”, “outer”, “above”, “below”, and the like, relate to the invention as it is shown in the drawing figures. However, it is to be understood that the invention can assume various alternative orientations and, accordingly, such terms are not to be considered as limiting.

Unless otherwise indicated, all numbers expressing quantities of ingredients, reaction conditions, processing parameters, physical characteristics, dimensions, and the like used in the specification and claims are to be understood as modified in all instances by the term “about.”

Unless otherwise indicated, all ranges or ratios disclosed herein are to be understood to encompass any and all subranges or subratios subsumed therein. For purposes of non-limiting illustration, a stated range or ratio of “1 to 10” should be considered to include any and all subranges between (and inclusive of) the minimum value of 1 and the maximum value of 10; that is, all subranges or subratios beginning with a minimum value of 1 or more and ending with a maximum value of 10 or less, such as but not limited to, 1 to 3.3, 4.7 to 7.5, 5.5 to 10, and the like.

As used herein, the articles “a,” “an,” and “the” include plural referents unless otherwise expressly and unequivocally limited to one referent.

As used herein recitations of “includes” and “including” mean “comprises” and “comprising,” unless otherwise indicated.

As used herein, the term “hydrocarbon” means materials that only include carbon and hydrogen, and which are free of other atoms, such as, but not limited to, oxygen, sulfur, and nitrogen.

The halogenated hydrocarbons of the present invention include at least one halogen that is covalently bonded to an sp² hybridized carbon. Correspondingly, the halogenated hydrocarbons of the present invention include at least one ethylenically unsaturated group, and at least one halogen is covalently bonded to an sp² hybridized carbon of an ethylenically unsaturated group. Further correspondingly, the halogenated hydrocarbons of the present invention include at least one non-aromatic ethylenically unsaturated group, and at least one halogen is covalently bonded to an sp² carbon of a non-aromatic ethylenically unsaturated group. With some embodiments, each ethylenically unsaturated group, of the halogenated hydrocarbons of the present invention, is independently selected from terminal ethylenically unsaturated groups and internal ethylenically unsaturated groups.

The halogenated hydrocarbons, with some embodiments of the present invention, are each independently selected from: halogenated linear or branched alkenes; halogenated cycloalkenes; an aromatic group having at least one halogenated linear or branched alkenyl group; an aromatic group having at least one halogenated cycloalkene group; and combinations thereof.

As used herein, recitations of “linear or branched,” such as, but not limited to, halogenated linear or branched alkene, are herein understood to include, for purposes of non-limiting illustration: compounds and groups that are linear, such as halogenated linear C₂-C₂₀ alkenes or linear C₂-C₂₀ alkenyl groups; and groups that are appropriately branched, such as, but not limited to, halogenated branched C₃-C₂₀ alkenes or halogenated branched C₃-C₂₀ alkenyl groups.

As used herein, the term “alkene” and related terms, such as “alkene groups” and “alkenyl groups” means groups which: are not aromatic; and include at least two carbon atoms, such as 2 to 20 carbon atoms, such as C₂-C₂₀ alkenes, or C₂-C₁₀ alkenes, or C₂-C₆ alkenes; are linear or branched; and include one or more interior and/or terminal alkene (or alkenyl) groups. Examples of alkenes include, but are not limited to, ethene, n-propene, iso-propene, n-butene, iso-butene, sec-butene, t-butene, linear or branched pentene, linear or branched hexene, linear or branched heptene, linear or branched octene, linear or branched nonene, linear or branched decene, linear or branched undecene, linear or branched dodecene, linear or branched tridecene, linear or branched tetradecene, linear or branched pentadecene, linear or branched hexadecene, linear or branched heptadecene, linear or branched octadecene, linear or branched nonadecene, and linear or branched eicosene.

As used herein, the term “cycloalkene” and related terms, such as “cycloalkenyl” means cyclic and non-aromatic hydrocarbons, which: include at least three carbon atoms, such as 3 to 20 carbon atoms, such as C₃-C₂₀ cycloalkenes, or C₃-C₁₀ cycloalkene, or C₃-C₈ cycloalkenes; and optionally include at least one fused ring selected from fused cycloalkane and fused cycloalkene. Examples of cycloalkenes include, but are not limited to: cyclopropene; cyclobutene; cyclopentene; cyclohexene; cycloheptene; cyclooctene; cyclononene; cyclodecene; cycloundecene; cyclododecene; bicyclo[2.2.1]heptene; octahydronaphthalene; dodecahydroanthracene; dodecahydrophenanthrene; and decahydro-1H-phenalene.

As used herein, the term “aromatic hydrocarbon” and related terms, such as “aromatic compound(s)” and “aromatic group(s)” means cyclic aromatic hydrocarbons (cyclic aromatic groups), which: include at least 5 carbon atoms, such as C₅-C₂₀ aromatic groups, or C₅-C₁₄ aromatic groups; and optionally include at least two fused aromatic hydrocarbon rings. Examples of aromatic hydrocarbons (aromatic compounds; aromatic groups) include, but are not limited to, benzene, naphthalene, anthracene, phenanthrene, and 1H-phenalene.

As used herein, the term “halogen” and related terms, such as “halogen group(s),” “halo group(s),” and/or “halogenated” means a single bonded halogen atom, such as selected from fluoro (F), chloro (CI), bromo (Br), and/or iodo (I).

As used herein, and unless otherwise explicitly stated, the term “hydrogen” and related terms, such as “hydrogen group(s)” means a single bonded hydrogen (—H).

All documents, such as but not limited to issued patents and patent applications, referred to herein, and unless otherwise indicated, are to be considered to be “incorporated by reference” in their entirety.

Each halogen of the halogenated hydrocarbons of the present invention are in each case independently selected from fluoro (F), chloro (Cl), bromo (Br), and iodo (I). In accordance with some embodiments, each halogen of the halogenated hydrocarbons of the present invention are in each case independently selected from chloro (Cl) and bromo (Br). In accordance with some embodiments, each halogen of the halogenated hydrocarbons of the present invention is chloro/chlorine (Cl), and the halogenated hydrocarbons are chlorinated hydrocarbons (where at least one chloro group (Cl) is covalently bonded to an sp² hybridized carbon).

In accordance with some embodiments of the present invention, the halogenated hydrocarbons are each independently selected from: halogenated linear or branched C₂-C₂₀ alkenes, or halogenated linear or branched C₂-C₁₀ alkenes, or halogenated linear or branched C₂-C₆ alkenes; halogenated C₃-C₂₀ cycloalkenes, or halogenated C₃-C₁₀ cycloalkenes, or halogenated C₃-C₈ cycloalkenes; aromatic group having at least one halogenated linear or branched C₂-C₂₀ alkenyl group, or aromatic group having at least one halogenated linear or branched C₂-C₁₀ alkenyl group, or aromatic group having at least one halogenated linear or branched C₂-C₆ alkenyl group; aromatic group having at least one halogenated C₃-C₂₀ cycloalkenyl group, or halogenated C₃-C₁₀ cycloalkenyl group, or halogenated C₃-C₈ cycloalkenyl group; and combinations thereof. With some embodiments, the aromatic group of the aromatic group having at least one halogenated linear or branched alkenyl group, and the aromatic group having at least one halogenated cycloalkenyl group, is in each case independently selected from benzene, naphthalene, and anthracene.

With some embodiments of the present invention, the halogenated hydrocarbons are each independently selected from: halogenated linear or branched C₂-C₂₀ alkenes, such as halogenated ethene, halogenated n-propene, halogenated iso-propene, halogenated n-butene, halogenated iso-butene, halogenated sec-butene, halogenated t-butene, halogenated linear or branched pentene, halogenated linear or branched hexene, and combinations thereof, where each halo group is independently selected from fluoro (F), chloro (CI), bromo (Br), and iodo (I).

The halogenated hydrocarbon, with some embodiments is a chlorinated hydrocarbon and is free of halogen groups other than chlorine groups, the first hydrogen halide content is a first hydrogen chloride content, the second hydrogen halide content is a second hydrogen chloride content, and the second hydrogen chloride content is less than the first hydrogen chloride content.

The chlorinated hydrocarbon, with some embodiments, is selected from chlorinated ethene (such as vinyl chloride), chlorinated n-propene, chlorinated iso-propene, chlorinated n-butene, chlorinated iso-butene, chlorinated sec-butene, chlorinated t-butene, chlorinated linear or branched pentene, chlorinated linear or branched hexene, and combinations of two or more thereof. The chlorinated hydrocarbon, with some embodiments, includes (or is) vinyl chloride.

The method of the present invention involves contacting together the first composition and a hydrotalcite material. As used herein and in the claims, the term “hydrotalcite material” means a material that includes, divalent metal cations, trivalent metal cations, hydroxides, at least one anion, and water, which is typically in the form of bound water. In accordance with some embodiments, for the hydrotalcite material: each divalent metal cation is independently selected from Mg²⁺, Ca²⁺, Ni²⁺, and Zn²⁺; each trivalent metal cation is independently selected from Al³⁺, Fe³⁺, Cr³⁺, and Co³⁺; and each anion is independently selected from CO₃²⁻, OH⁻, NO₃⁻, SO₄²⁻, and halides (F⁻, Cl⁻, Br⁻, and/or I⁻).

With some embodiments, the hydrotalcite material includes, divalent metal cations, trivalent metal cations, hydroxides, at least one anion, and water, where: each divalent metal is Mg²⁺; each trivalent metal is Al³⁺; each anion is CO₃²⁻; and where the water is in the form of bound water.

The hydrotalcite material, with some embodiments, is represented by the following Formula (A):

(M²⁺)₁₋ₓ(M³⁺)ₓ(OH)₂(Aⁿ⁻)_{x/n}·mH₂O (A)

With reference to Formula (A): M²⁺ is a divalent metal cation, which is selected in each case independently from Mg²⁺, Ca²⁺, Ni²⁺, and Zn²⁺; M³⁺ is a trivalent metal cation, which is selected in each case independently from Al³⁺, Fe³⁺, Cr³⁺, and Co³⁺; Aⁿ⁻ is an anion (where n is typically 1 or 2), which is selected from CO₃²⁻, OH⁻, NO₃_{^{−}}, SO₄²⁻, and halides (F⁻, Cl⁻, Br⁻, and/or I⁻). With further reference to Formula (A), and in accordance with some embodiments: x is 0.1 to 0.5; and m is 1 to 5.

With reference to Formula (A), and in accordance with some embodiments: M²⁺ is Mg²⁺; M³⁺ is Al³⁺; and Aⁿ⁻ is CO₃²⁻. With some embodiment, the hydrotalcite material has a layered hydrotalcite structure. Without intending to be bound by any theory, it is believed that the layered hydrotalcite structure includes spaced layers including magnesium hydroxide, with a layer including CO₃²⁻ and H₂O interposed between each pair of magnesium hydroxide containing layers.

The hydrotalcite material is solid and may have any suitable particle size or range of particle sizes, including average particle sizes. In accordance with some embodiments, the hydrotalcite material is solid and has an average particle size (such as a D50 particle size) of from 1 micrometer to 50 micrometers, or from 1 micrometers to 40 micrometers, or from 1 micrometers to 35 micrometers, or from 1 micrometers to 30 micrometers, or from 1 micrometers to 25 micrometers, or from 1 micrometers to 20 micrometers, or from 1 micrometers to 15 micrometers, or from 1 micrometers to 10 micrometers, including any combination of the recited upper and lower particle size values.

The hydrotalcite material is solid and typically has a granular or particulate form. In accordance with some embodiments, the hydrotalcite material is solid and has a granular form, such as, but not limited to, when used as (or in the form of) a fixed bed, such as within a fixed bed vessel. With some embodiments, the hydrotalcite material has an average particle size of from 0.1 mm to 75 mm, or 0.3 mm to 75 mm, or from 0.5 mm to 75 mm, or 1 mm to 75 mm, or from 1 mm to 50 mm, or from 1 mm to 25 mm, or from 0.3 mm to 10 mm, or from 0.5 mm to 10 mm, or from 1 mm to 10 mm, or from 2 mm to 8 mm, or from 3 mm to 7 mm.

In accordance with some embodiments, the hydrotalcite material is porous, and has a surface area (including the surface area of the pores) of from 8 to 500 m²/g, or from 12 to 100 m²/g, or from 40 to 60 m²/g.

The hydrotalcite material, with some embodiments, includes a support, such as silica and/or alumina. With some embodiments, the hydrotalcite material includes a binder, such as: an inorganic binder, bentonite or water glass; or an organic binder, such as polyvinyl alcohol and/or a surfactant.

With the method of the present invention, the first composition includes the halogenated hydrocarbon and a first hydrogen halide content. As used herein, recitations of “a first hydrogen halide content” also mean “a first amount of hydrogen halide.” The first hydrogen halide content can be referred to with regard to any appropriate unit, such as moles, volume, or weight. With some embodiments, the first hydrogen halide content has units of weight, such as grams or parts per million (ppm) where ppm is based on the total weight of the first composition. With some embodiments, the first hydrogen halide content is from 0.01 ppm to 500 ppm, or from 0.01 to 100 ppm, or from 0.1 ppm to 50 ppm, or from 0.5 ppm to 20 ppm, or from 1 ppm to 10 ppm.

The first composition, with some embodiments, is liquid. The first composition, with some embodiments, is liquid at ambient temperature and pressure. With some embodiments, the first composition is liquid under conditions of elevated pressure, such as from 25 psi to 300 psi, or from 25 psi to 240 psi. With some embodiments, the psi units are gauge, psig.

In accordance with the present method, the first composition and the hydrotalcite material are contacted together. The first composition and the hydrotalcite material can be contacted together using a batch process and/or a continuous process (such as introducing the first composition into a fixed bed containing the hydrotalcite material, as discussed in further detail herein). When contacted together using a batch process, the first composition and the hydrotalcite material are typically subjected to continuous stirring, such as with an impeller. The first composition and the hydrotalcite material can be contacted together under any appropriate temperature and pressure, such as ambient temperature and ambient pressure. With some embodiments, the first composition and the hydrotalcite material are contacted together at a temperature of from −20 °C to 300 °C, or from 30 °C to 200 °C, or from 50 °C to 177 °C, or from 60 °C to 125 °C, or from 70 °C to 110 °C, or from 80 °C to 100 °C, or from 85 °C to 95 °C, inclusive of the recited temperature values. With some additional embodiments, the first composition and the hydrotalcite material are contacted together under conditions of elevated pressure, such as from 25 psi to 300 psi, or from 30 psi to 200 psi, or from 40 psi to 100 psi.

In accordance with some embodiments, contacting together the first composition and the hydrotalcite material is conducted at: a temperature of from greater than 0 °C to 50 °C; and a pressure of from 25 psi to 300 psi.

In accordance with some further embodiments, the halogenated hydrocarbon includes vinyl chloride, and contacting together the first composition and the hydrotalcite material is conducted at: a temperature of from greater than 0 °C to 50 °C; and a pressure of from 25 psi to 300 psi.

In accordance with some embodiments, when contacted together, a weight ratio of the hydrotalcite material to the first hydrogen halide content is: at least 1:1, or at least 1.5:1, or at least 2.0:1; and less than or equal to 130:1, or less than or equal to 100:1, or less than or equal to 80:1, or less than or equal to 50:1, or less than or equal to 30:1, or less than or equal to 20:1, or less than or equal to 10:1, or less than or equal to 5:1. The weight ratio of the hydrotalcite material to the first hydrogen halide content, with some embodiments, can range between any combination of these lower and upper ratios (inclusive of the recited ratios). In accordance with some embodiments, when contacted together, the weight ratio of the hydrotalcite material to the first hydrogen halide content is from 1:1 to 130:1, or from 1:1 to 100:1, or from 1:1 to 80:1, or from 1:1 to 50:1, or from 1:1 to 30:1, or from 1:1 to 20:1, or from 1:1 to 10:1, or from 1:1 to 5:1.

Contacting together the first composition and the hydrotalcite material results in the formation of a second composition that includes the halogenated hydrocarbon and a second hydrogen halide content. The second hydrogen halide content is less than the first hydrogen halide content. As used herein, recitations of “a second hydrogen halide content” also mean “a second amount of hydrogen halide.” The second hydrogen halide content can be referred to with regard to any appropriate unit, such as moles, volume, or weight. With some embodiments, the second hydrogen halide content has units of weight, such as grams or parts per million (ppm) where ppm is based on the total weight of the second composition.

The second hydrogen halide content is less than the first hydrogen halide content, with the method of the present invention. With some embodiments, the second hydrogen halide content is at least 20% by weight less than the first hydrogen halide content, such as at least 30% by weight less than said first hydrogen halide content, or at least 40% by weight less than the first hydrogen halide content, or at least 50% by weight less than the first hydrogen halide content, at least 60% by weight less than the first hydrogen halide content, at least 70% by weight less than the first hydrogen halide content, or at least 80% by weight less than the first hydrogen halide content, or at least 90% by weight less than the first hydrogen halide content, or at least 95% by weight less than the first hydrogen halide content, or at least 98% by weight less than the first hydrogen halide content, or at least 99% by weight less than the first hydrogen halide content. In accordance with some embodiments, the second hydrogen halide content is 100% by weight less than the first hydrogen halide content.

The first hydrogen halide content and the second hydrogen halide content can be determined in accordance with art recognized methods, such as titration methods.

The first composition and the hydrotalcite material can be contacted together for any appropriate period of time, provided that the second hydrogen halide content is less than the first hydrogen halide content. With some embodiments the first composition and the hydrotalcite material are contacted together for a period of time that is at least sufficient to result in the second hydrogen halide content to be at least 20% by weight less than the first hydrogen halide contact. The first composition and the hydrotalcite material are contacted together, with some embodiments, for 1 minute to 24 hours, or from 2 minutes to 10 hours, or from 10 minutes to 6 hours.

The method of the present invention, with some embodiments, is conducted in the absence of liquid water. As used herein, the recitation of “conducted in the absence of liquid water” does not include bound water, such as bound water that is part of the hydrotalcite material. In accordance with some embodiments, the method of the present invention is conducted in the substantial absence of liquid water, such as liquid water being present in an amount of less than 100 ppm, or less than 50 ppm, or less than 25 ppm, or less than 10 ppm, or less than 5 ppm, or less than 2 ppm, or less than 1 ppm, or less than 0.5 ppm, or less than 0.1 ppm, in each case independently based on the first composition, and based on the second composition.

With some embodiments, contacting together the first composition and the hydrotalcite material is conducted in the presence of an organic solvent that is free of halogen groups. The organic solvent, with some embodiments, is provided or introduced separately from the first composition and the hydrotalcite material. With some embodiments, the organic solvent is: present as part of the first composition; and/or present with the hydrotalcite material. The organic solvent may be present in any appropriate amount, such as from 1 percent by weight to 70 percent by weight, or from 5 percent by weight to 60 percent by weight, or from 8 percent by weight to 50 percent by weight, or from 10 percent by weight to 45 percent by weight, the percent weights being based on the total weight of the first composition and the hydrotalcite material.

The organic solvent, with some embodiments, is selected from hydrocarbons that are free of halogen groups, such as, one or more linear or branched C₂-C₂₀ alkanes, and/or one or more C₃-C₂₀ cycloalkanes.

The method of the present invention involves isolating the second composition from the hydrotalcite material. The second composition can be isolated from the hydrotalcite in accordance with any appropriate method, such as, but not limited to, filtration and/or centrifugation. The isolated second composition, with some embodiments, is optionally subjected to art-recognized work-up procedures for purposes of further isolating and/or purifying the product halogenated hydrocarbon.

The method of the present invention can be conducted under batch conditions, continuous conditions, or a combination thereof. With some embodiments, the method of the present invention is conducted under batch conditions, which involves contacting together the first composition and the hydrotalcite material, and optionally an organic solvent in a suitable vessel, such as a reactor, to form a heterogeneous mixture. The heterogeneous mixture, with some embodiments, is subjected to agitation or stirring, such as by an impeller, and an optional increase in temperature for a period of time, during which and/or after-which samples are taken to determine the degree of (or extent to which) the second hydrogen halide content has been reduced relative to the first hydrogen halide content. When the second hydrogen halide content reaches a desired level (or level of reduction relative to the first hydrogen halide content) the second composition is isolated from the hydrotalcite material, such as by filtration.

The method of the present invention is, with some further embodiments, conducted under continuous conditions, which involves: (a) providing a first stream that includes the first composition, as described previously herein; and (b) providing a first fixed bed vessel including a first fixed bed that includes a hydrotalcite material. The method further includes, (c) introducing the feed stream into the first fixed bed vessel, and contacting together within the first fixed bed vessel the first composition and the hydrotalcite material. The method additionally includes, (d) withdrawing a product stream from the first fixed bed vessel, where the product stream includes a second composition that includes the halogenated hydrocarbon and has a second hydrogen halide content that is less than the first hydrogen halide content.

For purposes of non-limiting illustration, and in accordance with some embodiments, a method of treating a halogenated hydrocarbon is described with reference to FIG. 1. With reference to FIG. 1, there is schematically depicted a process assembly 3 that includes a first fixed bed vessel 11 that includes therein a first fixed bed 14 that includes a hydrotalcite material. First fixed bed vessel 11 is depicted with a partial cut-away thereof for purposes of illustrating the first fixed bed 14. With some embodiments, the first fixed bed 14 is defined by the hydrotalcite material. The first fixed bed vessel can be fabricated from any suitable material or combination of materials including, for example, metal (such as stainless steel), glass, plastics (including thermoplastics and/or crosslinked plastics), fiberglass filled plastics, and combinations thereof.

In accordance with some embodiments of the method of the present invention, there is provided a feed stream that includes a first composition including a halogenated hydrocarbon that includes at least one halogen group, where at least one halogen group is covalently bonded to an sp² hybridized carbon (as described previously herein), and where the first composition has a first hydrogen halide content. With further reference to FIG. 1, the feed stream is passed through conduit 17 and introduced into the first fixed bed vessel 11. Within the first fixed bed vessel 11, the first composition and the hydrotalcite material are contacted together, which results in the formation of the second composition. In FIG. 1 and FIG. 2, the flow direction of the various streams through the conduits is indicated by arrows.

With further reference to FIG. 1, a product stream that includes the second composition is withdrawn from the first fixed bed vessel 11 through outlet conduit 20, and with some embodiments, forwarded downstream to a storage vessel (not shown) and/or for further processing, such as work-up procedures. The second composition includes the halogenated hydrocarbon and a second hydrogen halide content that is less than the first hydrogen halide content.

With some embodiments, at least a portion of the product stream, which includes the second composition, is introduced back into the first fixed bed vessel. With reference to FIG. 1 and in accordance with some embodiments, at least a portion of the product stream is re-introduced into the first fixed bed vessel 11 through a recycle conduit 23 (or recycle circuit 23). The recycle conduit 23 has a first T-intersection 26 with outlet conduit 20, and a second T-intersection 29 with inlet conduit 17. The recycle conduit, with some embodiments, includes: a first recycle valve 32 that is positioned down-stream from the first T-intersection 26; a second recycle valve 35 that is positioned up-stream from second T-intersection 29; and optionally a pump 38 that is positioned inline between first recycle valve 32 and second recycle valve 35 (down-stream from first recycle valve 32 and up-stream from second recycle valve 35). Pump 38 includes an inlet 41 and an outlet 44.

In accordance with some embodiments, and with further reference to FIG. 1, inlet conduit 17 includes an inlet valve 47 that is positioned up-stream from second T-intersection 29, and outlet conduit 20 includes an outlet valve 50 that is positioned down-stream from first T-intersection 26. The process assembly 3 depicted in FIG. 1 can, with some embodiments, include more valves or fewer valves placed in alternate positions, such as at the first T-intersection 26 and/or at the second T-intersection 29.

In accordance with some embodiments and with further reference to FIG. 1, after introducing continuously the feed stream through inlet conduit 17 and into first fixed bed 11, and withdrawing continuously the product stream from the first fixed bed 11 through outlet conduit 20, inlet valve 47 and outlet valve 50 are each at least partially closed, first recycle valve 32 and second recycle valve 35 are at least partially opened, and pump 38 is activated. The sequence of valve closings, valve openings, and pump activation can be modified accordingly, and/or conducted manually and/or remotely, such as by one or more electrical connections (not shown). With outlet valve 50 at least partially closed, at least a portion of the product stream passes through first T-intersection 26, through first recycle valve 32, and through/into recycle conduit 23, into pump inlet 41, through pump 38, through pump outlet 44, through second recycle valve 35, through second T-intersection 29, through inlet conduit 17 and into first fixed bed 11. Recycling the product stream through the first fixed bed 11 is conducted, with some embodiments, for a period of time during which one or more samples are withdrawn and analyzed to determine whether the second hydrogen halide content is sufficiently less than the first hydrogen halide content. When the second hydrogen halide content has reached a desired level (of reduction relative to the first hydrogen halide content), the various valves can be adjusted so as to allow the product stream to be removed from the first fixed bed 11 through outlet conduit 20 and forwarded downstream for further processing and/or storage.

The method of the present invention further includes, with some embodiments: providing a second fixed bed vessel including a second fixed bed that includes a drying agent; introducing at least a portion of the product stream into the second fixed bed vessel where it contacts the drying agent, thereby removing at least a portion of residual water from the product stream, and forming a dried product stream; and removing the dried product stream from the second fixed bed vessel. With some embodiments, the drying agent in the second fixed bed vessel is selected from CaCl₂, alumina, silica, NaOH, molecular sieves, and combinations of two or more thereof. When the drying agent includes NaOH, the NaOH, with some embodiments, has a prill (or pellet) form.

With some embodiments, the product stream includes residual water in an amount of less than or equal to 1 percent by weight, or less than or equal to 0.5 percent by weight, or less than or equal to 0.25 percent by weight, or less than or equal to 0.1 percent by weight, or less than or equal to 0.05 percent by weight, or less than or equal to 0.01 percent by weight, the percent weights in each case being based on the total weight of the product stream.

With reference to FIG. 2, there is schematically depicted a process assembly 5, which includes portions of process assembly 3 of FIG. 1, and further includes a second fixed bed vessel 53 that includes a second fixed bed 56 including a drying agent (such as being composed of CaCl₂ with some embodiments). The CaCl₂) is, with some embodiments, present in particulate form. The particulate CaCl₂) can have any suitable particle sized, such as an average particle sized of from 1 mm to 50 mm, or from 6 mm to 13 mm. Second fixed bed vessel 53 is depicted with a partial cut-away thereof for purposes of illustrating the second fixed bed 56. With some embodiments, the second fixed bed 56 is defined by the drying agent, such as CaCl₂). The second fixed bed vessel 53 is positioned inline in (or with) recycle conduit 23, with some embodiments.

The process assembly 5, with some embodiments, includes a bypass conduit 59 that bypasses the second fixed bed vessel 53. The second fixed bed vessel 53 includes a second fixed bed vessel inlet valve 62 and a second fixed bed vessel outlet valve 65. Bypass conduit 59 has a first bypass T-intersection 68 with recycle conduit 23, and a second bypass T-intersection 71 with recycle conduit 23. The first bypass T-intersection 68 is positioned up-stream from the second fixed bed inlet valve 62, and the second bypass T-intersection 71 is positioned down-stream from the second fixed bed vessel outlet valve 65. Bypass conduit 59 includes a first bypass valve 74 and a second bypass valve 77. First bypass valve 74 is positioned down-stream from first bypass T-intersection 68, and second bypass valve 77 is positioned up-stream from second bypass T-intersection 71.

With reference to FIG. 2, and in accordance with some embodiments, with the feed stream introduced continuously through inlet conduit 17 and into/through first fixed bed 11: inlet valve 47 and outlet valve 50 of the first fixed bed 11 are each at least partially closed; first recycle valve 32 and second recycle valve 35 are each at least partially opened; second fixed bed inlet valve 62 and second fixed bed outlet valve 65 are each at least partially opened; and first bypass valve 74 and second bypass valve 77 are each closed. With the valves of process assembly 5 of FIG. 2 so arranged, at least a portion of the product stream passes through recycle conduit 23 and into second fixed bed vessel 53 where it contacts second fixed bed 56 including CaCl₂), which results in the formation of a dried product stream that is removed from second fixed bed 56 through second fixed bed vessel outlet valve 65.

The dried product stream removed from the second fixed bed vessel 53 can be forwarded downstream for storage and/or further processing through one or more further conduits, such as through conduit 36, with valve 35 appropriately turned (or adjusted). With some embodiments, the method of the present invention further includes introducing at least a portion of the dried product stream into the first fixed bed vessel. With reference to FIG. 2, and in accordance with some embodiments, the dried product stream is forwarded through pump 38, through second recycle valve 35, through second T-intersection 29, through inlet conduit 17 and into first fixed bed vessel 11. The dried product stream can be passed numerous times through the first fixed bed 11 and the second fixed bed 53 for purposes of further drying the product stream. With the various valves adjusted accordingly (such as described previously herein), the dried product stream is passed through outlet conduit 20 and forwarded downstream for further processing and/or storage.

With reference to FIG. 2, the second fixed bed vessel 53 can be bypassed, with some embodiments, by: closing the second fixed bed vessel inlet valve 62 and closing the second fixed bed outlet valve 65; and opening the first bypass valve 74 and opening the second bypass valve 77. With the valves so arranged, the recycle conduit and the bypass conduit 59 together serve as a combined recycle conduit 23-59 that bypasses the second fixed bed vessel 53.

With some embodiments, the feed stream has a total (or maximum) volume that correspondingly provides a total (or maximum) weight of the first hydrogen halide content. The weight ratio of the hydrotalcite material (of the first fixed bed within the first fixed bed vessel) to the total weight of the first hydrogen halide content is, with some embodiments: at least 1:1, or at least 1.5:1, or at least 2.0:1; and less than or equal to 130:1, or less than or equal to 100:1, or less than or equal to 80:1, or less than or equal to 50:1, or less than or equal to 30:1, or less than or equal to 20:1, or less than or equal to 10:1, or less than or equal to 5:1. The weight ratio of the hydrotalcite material (of the first fixed bed within the first fixed bed vessel) to the total weight of the first hydrogen halide content, with some embodiments, can range between any combination of these lower and upper ratios (inclusive of the recited ratios). In accordance with some embodiments, the weight ratio of the hydrotalcite material (of the first fixed bed within the first fixed bed vessel) to the total weight of the first hydrogen halide content is, from 1:1 to 130:1, or from 1:1 to 100:1, or from 1:1 to 80:1, or from 1:1 to 50:1, or from 1:1 to 30:1, or from 1:1 to 20:1, or from 1:1 to 10:1, or from 1:1 to 5:1.

Contacting together the first composition and the hydrotalcite material (of the first fixed bed within the first fixed bed vessel) is conducted, with some embodiments, at a temperature of from −20 °C to 300 °C, or from 30 °C to 200 °C, or from 50 °C to 177 °C, or from 60 °C to 125 °C, or from 70 °C to 110 °C, or from 80 °C to 100 °C, or from 85 °C to 95 °C, inclusive of the recited temperature values. The temperature at which the first composition and the hydrotalcite material are contacted within the first fixed bed vessel is, with some embodiments, controlled by a heat exchange jacket (not shown) that envelopes at least a portion of the exterior of the first fixed bed vessel 11, and/or by passing the feed stream through a heat exchanger (not shown). With some embodiments, recycle conduit 23 includes a heat exchanger (not shown) that maintains the temperature of the recycled feed/product stream within a desired (or preselected) range.

Contacting together the first composition and the hydrotalcite material (of the first fixed bed within the first fixed bed vessel) is conducted, with some embodiments, at ambient pressure. With some further embodiments, contacting together the first composition and the hydrotalcite material (of the first fixed bed within the first fixed bed vessel) is conducted under conditions of elevated pressure, such as from 25 psi to 300 psi, or from 30 psi to 200 psi, or from 40 psi to 100 psi.

According to some embodiments, the halogenated hydrocarbon (of the first and second compositions) is a chlorinated hydrocarbon and is free of halogen groups other than chlorine groups, the first hydrogen halide content is a first hydrogen chloride content, the second hydrogen halide content is a second hydrogen chloride content, and the second hydrogen chloride content is less than the first hydrogen chloride content.

The chlorinated hydrocarbon (of the first and second compositions), with some embodiments, is selected from chlorinated ethene (such as vinyl chloride), chlorinated n-propene, chlorinated iso-propene, chlorinated n-butene, chlorinated iso-butene, chlorinated sec-butene, chlorinated t-butene, chlorinated linear or branched pentene, chlorinated linear or branched hexene, and combinations of two or more thereof.

The chlorinated hydrocarbon, with some embodiments, includes (or is) vinyl chloride.

The present invention can be further characterized by one or more of the following non-limiting clauses.

Clause 1: A method of treating a halogenated hydrocarbon, comprising:
(a) providing a first composition comprising a halogenated hydrocarbon comprising at least one halogen group, wherein at least one halogen group is covalently bonded to an sp² hybridized carbon, wherein said first composition has a first hydrogen halide content;
(b) contacting together said first composition and a hydrotalcite material, thereby forming a second composition comprising said halogenated hydrocarbon and having a second hydrogen halide content, wherein said second hydrogen halide content is less than said first hydrogen halide content; and
(c) isolating said second composition from said hydrotalcite material.

Clause 2: The method of clause 1 wherein, a weight ratio of said hydrotalcite material to said first hydrogen halide content is from 1:1 to 130:1, or from 1:1 to 100:1, or from 1:1 to 80:1, or from 1:1 to 50:1, or from 1:1 to 30:1, or from 1:1 to 20:1, or from 1:1 to 10:1, or from 1:1 to 5:1.

Clause 3: The method of clause 1 or clause 2 wherein, said hydrotalcite material has an average particle size of from 0.3 to 10 mm, or from 0.5 mm to 10 mm, or from 1 mm to 10 mm, or from 2 mm to 8 mm, or from 3 mm to 7 mm.

Clause 4: The method of any one of clauses 1 to 3 wherein, said hydrotalcite material comprises, divalent metal cations, trivalent metal cations, hydroxides, at least one anion, and water, wherein for said hydrotalcite material, each divalent metal is Mg²⁺, each trivalent metal is Al³⁺, and each anion is CO₃²⁻.

Clause 5: The method of any one of clauses 1 to 4 wherein, contacting together said first composition and said hydrotalcite material is conducted at a temperature of from −20 °C to 300 °C, or from 30 °C to 200 °C, or from 50 °C to 177 °C, or from 60 °C to 125 °C, or from 70 °C to 110 °C, or from 80 °C to 100 °C, or from 85 °C to 95 °C.

Clause 6: The method of any one of clauses 1 to 5 wherein, said method is conducted in the absence of liquid water.

Clause 7: The method of any one of clauses 1 to 6 wherein, said second hydrogen halide content is at least 20% by weight less than said first hydrogen halide content, or at least 40% by weight less than the first hydrogen halide content, or at least 50% by weight less than the first hydrogen halide content, at least 60% by weight less than the first hydrogen halide content, at least 70% by weight less than the first hydrogen halide content, or at least 80% by weight less than the first hydrogen halide content, or at least 90% by weight less than the first hydrogen halide content, or at least 95% by weight less than the first hydrogen halide content, or at least 98% by weight less than the first hydrogen halide content, or at least 99% by weight less than the first hydrogen halide content, or 100% by weight less than the first hydrogen halide content.

Clause 8: The method of any one of clauses 1 to 7 wherein, said halogenated hydrocarbon is selected from the group consisting of: halogenated linear or branched alkenes; halogenated cycloalkenes; aromatic group having at least one halogenated linear or branched alkenyl group; aromatic group having at least one halogenated cycloalkenyl group; and combinations thereof.

Clause 9: The method of any one of clauses 1 to 8 wherein, said halogenated hydrocarbon is selected from the group consisting of: halogenated linear or branched C₂-C₂₀ alkenes; halogenated C₃-C₂₀ cycloalkenes; aromatic group having at least one halogenated linear or branched C₂-C₂₀ alkenyl group; aromatic group having at least one halogenated C₃-C₂₀ cycloalkenyl group; and combinations thereof.

Clause 10: The method of any one of clauses 1 to 9 wherein, said halogenated hydrocarbon is a chlorinated hydrocarbon and is free of halogen groups other than chlorine groups, said first hydrogen halide content is a first hydrogen chloride content, said second hydrogen halide content is a second hydrogen chloride content, and said second hydrogen chloride content is less than said first hydrogen chloride content.

Clause 11: The method of any one of clauses 1 to 10 wherein, said halogenated hydrocarbon comprises vinyl chloride.

Clause 12: The method of any one of clauses 1 to 11 wherein, contacting together said first composition and said hydrotalcite material is conducted at: a temperature of −20 °C to 300 °C, or from 30 °C to 200 °C, or from 50 °C to 177 °C, or from 60 °C to 125 °C, or from 70 °C to 110 °C, or from 80 °C to 100 °C, or from 85 °C to 95 °C; and a pressure of from 25 psi to 300 psi, or from 30 psi to 200 psi, or from 40 psi to 100 psi.

Clause 13: The method of any one of clauses 1 to 12 wherein, contacting together said first composition and said hydrotalcite material is conducted in the presence of an organic solvent that is free of halogen groups.

Clause 14: A method of treating a halogenated hydrocarbon, comprising:
(a) providing a feed stream comprising a first composition comprising a halogenated hydrocarbon comprising at least one halogen group, wherein at least one halogen group is covalently bonded to an sp² hybridized carbon, wherein said first composition has a first hydrogen halide content;
(b) providing a first fixed bed vessel comprising a first fixed bed comprising a hydrotalcite material;
(c) introducing said feed stream into said first fixed bed vessel, and contacting together within said first fixed bed vessel said first composition and said hydrotalcite material; and
(d) withdrawing a product stream from said first fixed bed vessel, wherein said product stream comprises a second composition comprising said halogenated hydrocarbon having a second hydrogen halide content, wherein said second hydrogen halide content is less than said first hydrogen halide content.

Clause 15: The method of clause 14 wherein, at least a portion of said product stream is introduced into said first fixed bed vessel.

Clause 16: The method of clause 14 or clause 15 further comprising, providing a second fixed bed vessel comprising a second fixed bed comprising a drying agent, introducing at least a portion of said product stream into said second fixed bed vessel, thereby removing at least a portion of residual water from said product stream, and forming a dried product stream, and removing said dried product stream from said second fixed bed vessel.

Clause 17: The method of clause 15 wherein, said drying agent is selected from CaCl₂, alumina, silica, NaOH, molecular sieves, and combinations thereof.

Clause 18: The method of clause 16 or clause 17 further comprising, introducing at least a portion of said dried product stream into said first fixed bed vessel.

Clause 19: The method of any one of clauses 14 to 18 wherein, said feed stream has a total (or maximum) volume providing a total (or maximum) weight of said first hydrogen halide content, and a weight ratio of said hydrotalcite material to said total weight of said first hydrogen halide content is from 1:1 to 130:1, or from 1:1 to 100:1, or from 1:1 to 80:1, or from 1:1 to 50:1, or from 1:1 to 30:1, or from 1:1 to 20:1, or from 1:1 to 10:1, or from 1:1 to 5:1.

Clause 20: The method of any one of clauses 14 to 19 wherein, contacting together, within said first fixed bed vessel, said first composition and said hydrotalcite material, is conducted at a temperature of from −20 °C to 300 °C, or from 30 °C to 200 °C, or from 50 °C to 177 °C, or from 60 °C to 125 °C, or from 70 °C to 110 °C, or from 80 °C to 100 °C, or from 85 °C to 95 °C.

Clause 21: The method of any one of clauses 14 to 20 wherein, said halogenated hydrocarbon is a chlorinated hydrocarbon and is free of halogen groups other than chlorine groups, said first hydrogen halide content is a first hydrogen chloride content, said second hydrogen halide content is a second hydrogen chloride content, and said second hydrogen chloride content is less than said first hydrogen chloride content.

Clause 22: The method of any one of clauses 14 to 21 wherein, said halogenated hydrocarbon comprises vinyl chloride.

Clause 23: The method of any one of clauses 14 to 22 wherein, contacting together said first composition and said hydrotalcite material is conducted at: a temperature of −20 °C to 300 °C, or from 30 °C to 200 °C, or from 50 °C to 177 °C, or from 60 °C to 125 °C, or from 70 °C to 110 °C, or from 80 °C to 100 °C, or from 85 °C to 95 °C; and a pressure of from 25 psi to 300 psi, or from 30 psi to 200 psi, or from 40 psi to 100 psi.

The present invention is more particularly described in the following examples, which are intended as illustrative only, since numerous modifications and variations therein will be apparent to those skilled in the art.

### EXAMPLES

In Part-1 of the following examples, the continuous treatment of vinyl chloride monomer is described. In Part-2 of the following examples, the treatment of 1,2-dichloroethane is described.

### Part-1

### Example A

A stainless steel pipe reactor having a flanged inlet section and a flanged outlet section was used. The stainless steel pipe reactor had a length of 105 cm and an inner diameter of 2.8 cm. The stainless steel pipe reactor was filled with 350.4 grams of calcined hydrotalcite material (obtained from Kisuma Chemicals BV, having the designation of KW-500-G7). The calcined hydrotalcite material had: an MgO to Al₂O₃ ratio of from 5.7:1 to 6.2:1; a BET surface area of from 60 to 100 m²/g; and an average particle size of 0.7 mm. Plastic mesh screens were placed over the flanged inlet and flanged outlet, prior to mating with inlet and outlet conduits. The mesh size (200 micrometers) of the plastic mesh screens was selected so as to contain the calcined hydrotalcite within the pipe reactor, while allowing feed vinyl chloride monomer to flow therethrough. Feed vinyl chloride monomer to which had been added HCl was provided. The feed vinyl chloride monomer was determined to contain 60 ppm HCl (by potentiometric titration). The feed vinyl chloride monomer was passed continuously through the calcined hydrotalcite containing pipe reactor at a rate of 0.264 gallons/minute (0.131 liters/min) and a pressure of 145 psi (999.7 kPa). A total of 33 pounds (15 Kg) of treated vinyl chloride monomer was collected from the outlet conduit of the calcined hydrotalcite containing pipe reactor and contained in a pressurized cylinder. Breakthrough was not observed during the course of this Example A. The treated vinyl chloride monomer was determined to contain 0.5 ppm HCl (by potentiometric titration), representing a 99.17% by weight reduction in HCl content relative to the feed vinyl chloride monomer.

### Example B

A carbon steel pipe reactor having a flanged inlet section and a flanged outlet section was used. The carbon steel pipe reactor had an inner diameter of 3 inches (7.62 cm) and a length of fourteen inches (35.56 cm). The carbon steel pipe reactor was filled with 2.01 pounds (911.7 grams) of calcined hydrotalcite material (obtained from Kisuma Chemicals BV, having the designation of KW-500-G7). The calcined hydrotalcite material had: an MgO to Al₂O₃ ratio of from 5.7:1 to 6.2:1; a BET surface area of from 60 to 100 m²/g; and an average particle size of 0.7 mm. Stainless steel mesh screens were placed over the flanged inlet and flanged outlet, prior to mating with inlet and outlet conduits. The mesh size of the stainless steel screens was selected so as to contain the calcined hydrotalcite within the pipe reactor, while allowing feed vinyl chloride monomer to flow therethrough. The inlet conduit included a flow meter. Feed vinyl chloride monomer was provided under pressure. The feed vinyl chloride monomer was determined to have a pH of 2.71 and contain 71 ppm HCl (using a temperature controlled aqueous pH probe). The feed vinyl chloride monomer was passed through the calcined hydrotalcite containing pipe reactor at a rate of 1 gallon per minute (3.79 liters per minute) and a pressure of 240 psig (1654.7 kPa). Treated vinyl chloride monomer samples were collected from the outlet conduit immediately after starting the feed stream and every thirty minutes thereafter. The treated vinyl chloride monomer samples were collected in pressurized 300 ml cylinders. After three hours, treated vinyl chloride monomer samples were collected every hour until breakthrough was observed (when the pH dropped, and the HCL content of the treated samples significantly increased, indicating that the calcined hydrotalcite material in the reactor could no longer sufficiently remove HCl from the feed). The treated vinyl chloride monomer samples were analyzed for the presence or absence of HCl using a temperature controlled aqueous pH probe, the results of which are summarized in the following Table 1.

TABLE 1

| Time | Rate | | [H⁺] |
|---|---|---|---|
| (mins) | (g/min) | pH | ppm* |
| 0 | 0 | 2.71 | 71.00 |
| 4 | 1 | 3.19 | 23.00 |
| 30 | 1 | 6.94 | 0.00 |
| 60 | 1 | 7.01 | 0.00 |
| 90 | 1 | 7.54 | 0.00 |
| 120 | 1 | 7.66 | 0.00 |
| 150 | 1 | 7.55 | 0.00 |
| 180 | 1 | 7.55 | 0.00 |
| 240 | 1 | 7.54 | 0.00 |
| 300 | 1 | 2.86 | 50.34 |
| 360 | 1 | 2.81 | 56.48 |

With reference to Table 1, the HCl content of the treated vinyl chloride stream was observed to drop to zero at 30 minutes. A breakthrough was observed at 5 hours (occurring between 4 and 5 hours), after about 2400 pounds (1089 Kg) of feed vinyl chloride monomer had been passed through the calcined hydrotalcite containing pipe reactor. After stopping the introduction of feed vinyl chloride monomer into the inlet conduit, the calcined hydrotalcite containing pipe reactor was purged by introducing nitrogen gas through the inlet conduit, for purposes of removing any trace vinyl chloride monomer from the hydrotalcite. The nitrogen purged calcined hydrotalcite was recovered from the carbon steel pipe reactor, and was visually observed to be structurally unchanged, indicating that it had not undergone structural damage during passing feed vinyl chloride monomer therethrough under pressure.

### Part-2

For each of Examples 1-8, a first composition was prepared by mixing together 1000.0 grams of 1,2-dichloroethane (from Aldrich) and 0.0257 g of 12M HCl (from Fischer Scientific) in a glass container that was sealed with a polyethylene-lined cap and vigorously shaken for 60 seconds, after which the contents were observed to be clear and colorless, with no observable haze or presence of a second phase. Each first composition was titrated and determined to include 9.286 ppm of HCl as a first hydrogen chloride content.

For each of Examples 9-16, a first sample composition was prepared by mixing together 1000.0 grams of 1,2-dichloroethane (from Aldrich) and 0.1590 g of 12M HCl (from Fischer) in a glass container that was sealed with a polyethylene-lined cap and vigorously shaken for 60 seconds, after which the contents were observed to be clear and colorless, with no observable haze or presence of a second phase. The first sample composition was titrated and determined to initially include 46.177 ppm of HCl as a first hydrogen chloride content. Each first composition was drawn from the first sample composition.

With Examples 1-16, as summarized in the following Table 2, a four ounce glass bottle was charged with the recited amounts of the first composition and hydrotalcite (from Kisuma Chemicals B.V.) and included a magnetic stir bar. The bottle was immediately sealed with a polyethylene-lined cap, and mixed on a magnetic stir plate at room temperature for one hour. The contents of the bottle were filtered through a 13 mm diameter 0.45 micron PVDF syringe filter to separate the second composition from the hydrotalcite. A portion of the second composition was titrated to determine the second hydrogen chloride content.

TABLE 2

| | | First | | | | Second |
|---|---|---|---|---|---|---|
| | First | HCl | | HT/First | Smpl. | HCl |
| | Composition | Content | HT^{(a)} | HCl | Sz.^{(c)} | Content |
| Example | (g) | (ppm) | (g) | Content^{(b)} | (g) | (ppm) |
| 1 | 100.03 | 9.286 | KCEX24003^{(d)} | 28.11:1 | 36.0044 | 0 |
| | | | 0.0261 | | | |
| 2 | 100.06 | 9.286 | KCEX24003^{(d)} | 114.04:1 | 36.5324 | 0 |
| | | | 0.1059 | | | |
| 3 | 100.11 | 9.286 | KCEX24005^{(e)} | 8.94:1 | 39.9582 | 0 |
| | | | 0.0083 | | | |
| 4 | 99.98 | 9.286 | KCEX24005^{(e)} | 30.37:1 | 37.1317 | 0 |
| | | | 0.0282 | | | |
| 5 | 99.99 | 9.286 | KCEX24005^{(e)} | 123.09:1 | 40.6432 | 0 |
| | | | 0.1143 | | | |
| 6 | 100.09 | 9.286 | KCEX24006^{(f)} | 6.99:1 | 42.8265 | 0 |
| | | | 0.0065 | | | |
| 7 | 100.02 | 9.286 | KCEX24006^{(f)} | 29.94:1 | 43.2118 | 0 |
| | | | 0.0278 | | | |
| 8 | 71.95 | 9.286 | KCEX24006^{(f)} | 76.24:1 | 44.5967 | 0 |
| | | | 0.0708 | | | |
| 9 | 100.02 | 46.177 | KCEX24004^{(g)} | 7.69:1 | 41.3166 | 0 |
| | | | 0.0355 | | | |
| 10 | 100.05 | 46.177 | KCEX24004^{(g)} | 30.49:1 | 40.2818 | 0 |
| | | | 0.1408 | | | |
| 11 | 100.01 | 46.177 | KCEX24004^{(g)} | 121.60:1 | 46.3470 | 0 |
| | | | 0.5615 | | | |
| 12 | 100.06 | 46.177 | KCEX24004^{(g)} | 3.92:1 | 46.4494 | 0 |
| | | | 0.0181 | | | |
| 13 | 100.04 | 24.127 | KCEX24004^{(g)} | 1.95:1 | 43.0247 | 5.803^{(h)} |
| | | | 0.0090 | | | |
| 14 | 100.06 | 24.127 | KCEX24003^{(d)} | 2.25:1 | 40.5539 | 8.422⁽ⁱ⁾ |
| | | | 0.0104 | | | |
| 15 | 100.02 | 24.127 | KCEX24005^{(e)} | 2.17:1 | 43.5309 | 5.233^{(j)} |
| | | | 0.0100 | | | |
| 16 | 100.02 | 24.127 | KCEX24006^{(f)} | 2.27:1 | 46.9312 | 3.715^{(k)} |
| | | | 0.0105 | | | |

^{(a)}Hydrotalcite designation and grams used. All hydrotalcite samples were obtained from Kisuma Chemicals B.V.
^{(b)}Weight ratio of hydrotalcite to the first HCl content.
^{(c)}Sample Size: the amount, in grams, of the filtered hydrotalcite treated sample (the second composition) that was titrated to determine the Second HCl Content.
^{(d)}KCEX24003 hydrotalcite, in each case, had: an MgO to Al₂O₃ ratio of from 4.0:1 to 4.3:1; a BET surface area of from 80 to 120 m²/g; a D50 particle size of 7 micrometers (with 70% by weight thereof being greater than 5 micrometers); and was in the form of a powder.
^{(e)}KCEX24005 hydrotalcite, in each case, had: an MgO to Al₂O₃ ratio of from 5.7:1 to 6.2:1; a BET surface area of from 90 to 130 m²/g; a D50 particle size of 7 micrometers (with 70% by weight thereof being greater than 5 micrometers); and was in the form of a powder.
^{(f)}KCEX24006 hydrotalcite, in each case, had: an MgO to Al₂O₃ ratio of from 7.6:1 to 8.3:1; a BET surface area of from 50 to 60 m²/g; a D50 particle size of 6.5 micrometers (with 65% by weight thereof being greater than 5 micrometers); and was in the form of a powder.
^{(g)}KCEX24004 hydrotalcite, in each case, was calcined KCEX24003 hydrotalcite, and had: an MgO to Al₂O₃ ratio of from 4.1:1 to 4.6:1; a BET particle size of 200 m²/g; a D50 particle size of 20 micrometers (with 78% by weight thereof being greater than 5 micrometers); and was in the form of a powder.
^{(h)}For Example 13: 5.803 ppm HCl remaining corresponded to 18.3240 ppm of HCl that was neutralized, which provides to a ratio of (g HCl neutralized/g HT) of 0.2037. Examples 13-16 were run two days after Example 12 was run, at which point the remaining first sample composition was again titrated and the First HCl Content was determined to be 24.127 ppm. The reduced First HCl Content is thought to be due to gaseous HCl collecting in the headspace of the sample container, which was lost (escaped) when the sample container was opened to withdraw portions for Examples 13-16.
⁽ⁱ⁾For Example 14: 8.422 ppm HCl remaining corresponded to 15.7050 ppm of HCl that was neutralized, which provides to a ratio of (g HCl neutralized/g HT) of 0.1511.
^{(j)}For Example 15: 5.233 ppm HCl remaining corresponded to 18.8940 ppm of HCl that was neutralized, which provides to a ratio of (g HCl neutralized/g HT) of 0.1511.
^{(k)}For Example 16: 3.715 ppm HCl remaining corresponded to 20.412 ppm of HCl that was neutralized, which provides a ratio of (g HCl neutralized/g HT) of 0.1944.

The present invention has been described with reference to specific details of particular embodiments thereof. However, it is not intended that such details be regarded as limitations upon the scope of the invention except insofar as and to the extent that they are included in the accompanying claims.

What is claimed is:

1. A method of treating a halogenated hydrocarbon, comprising:
(a) providing a first composition comprising a halogenated hydrocarbon comprising at least one halogen group, wherein at least one halogen group is covalently bonded to an sp² hybridized carbon, wherein said first composition has a first hydrogen halide content;
(b) contacting together said first composition and a hydrotalcite material, thereby forming a second composition comprising said halogenated hydrocarbon and having a second hydrogen halide content, wherein said second hydrogen halide content is less than said first hydrogen halide content; and
(c) isolating said second composition from said hydrotalcite material.

2. The method of claim 1, wherein a weight ratio of said hydrotalcite material to said first hydrogen halide content is from 1:1 to 130:1.

3. The method of claim 1, wherein said hydrotalcite material has an average particle size of from 0.3 mm to 10 mm.

4. The method of claim 1, wherein said hydrotalcite material comprises, divalent metal cations, trivalent metal cations, hydroxides, at least one anion, and water,
wherein for said hydrotalcite material, each divalent metal is Mg²⁺, each trivalent metal is Al³⁺, and each anion is CO₃²⁻.

5. The method of claim 1, wherein contacting together said first composition and said hydrotalcite material is conducted at a temperature of from −20 °C to 300 °C.

6. The method of claim 1, wherein said method is conducted in the absence of liquid water.

7. The method of claim 1, wherein said second hydrogen halide content is at least 20% by weight less than said first hydrogen halide content.

8. The method of claim 1, wherein said halogenated hydrocarbon is selected from the group consisting of: halogenated linear or branched alkenes; halogenated cycloalkenes; aromatic group having at least one halogenated linear or branched alkenyl group; aromatic group having at least one halogenated cycloalkenyl group; and combinations thereof.

9. The method of claim 1, wherein said halogenated hydrocarbon is a chlorinated hydrocarbon and is free of halogen groups other than chlorine groups, said first hydrogen halide content is a first hydrogen chloride content, said second hydrogen halide content is a second hydrogen chloride content, and said second hydrogen chloride content is less than said first hydrogen chloride content.

10. The method of claim 9, wherein said chlorinated hydrocarbon comprises vinyl chloride.

11. The method of claim 10, wherein contacting together said first composition and said hydrotalcite material is conducted at: a temperature of from greater than 0 °C to 50 °C; and a pressure of from 25 psi to 300 psi.

12. The method of claim 1, wherein contacting together said first composition and said hydrotalcite material is conducted in the presence of an organic solvent that is free of halogen groups.

13. A method of treating a halogenated hydrocarbon, comprising:
(a) providing a feed stream comprising a first composition comprising a halogenated hydrocarbon comprising at least one halogen group, wherein at least one halogen group is covalently bonded to an sp² hybridized carbon, wherein said first composition has a first hydrogen halide content;
(b) providing a first fixed bed vessel comprising a first fixed bed comprising a hydrotalcite material;
(c) introducing said feed stream into said first fixed bed vessel, and contacting together within said first fixed bed vessel said first composition and said hydrotalcite material; and
(d) withdrawing a product stream from said first fixed bed vessel, wherein said product stream comprises a second composition comprising said halogenated hydrocarbon having a second hydrogen halide content, wherein said second hydrogen halide content is less than said first hydrogen halide content.

14. The method of claim 13, wherein at least a portion of said product stream is introduced into said first fixed bed vessel.

15. The method of claim 13, further comprising:
providing a second fixed bed vessel comprising a second fixed bed comprising a drying agent,
introducing at least a portion of said product stream into said second fixed bed vessel, thereby removing at least a portion of residual water from said product stream, and forming a dried product stream, and
removing said dried product stream from said second fixed bed vessel.

16. The method of claim 15, wherein said drying agent is selected from CaCl₂, alumina, silica, NaOH, molecular sieves, and combinations thereof.

17. The method of claim 15, further comprising:
introducing at least a portion of said dried product stream into said first fixed bed vessel.

18. The method of claim 13, wherein,
said feed stream has a total volume providing a total weight of said first hydrogen halide content, and
a weight ratio of said hydrotalcite material to said total weight of said first hydrogen halide content is from 1:1 to 130:1.

19. The method of claim 13, wherein contacting together, within said first fixed bed vessel, said first composition and said hydrotalcite material, is conducted at a temperature of from −20 °C to 300 °C.

20. The method of claim 13, wherein said halogenated hydrocarbon is a chlorinated hydrocarbon and is free of halogen groups other than chlorine groups, said first hydrogen halide content is a first hydrogen chloride content, said second hydrogen halide content is a second hydrogen chloride content, and said second hydrogen chloride content is less than said first hydrogen chloride content.

21. The method of claim 20, wherein said chlorinated hydrocarbon comprises vinyl chloride.
